(19) United States
(12) Patent Application Publication
(10) Pub. No.: US 2024/0141290 A1
(43) Pub. Date: May 2, 2024

(54) METHODS AND SYSTEMS FOR SEPARATING SPERM CELLS FOR ASSESSING AND/OR QUANTIFYING SPERM CELL SUBPOPULATIONS BEARING A SPECIFIC GENETIC SIGNATURE

(71) Applicant: ABS Global, Inc., DeForest, WI (US)

(72) Inventor: Michael Botts, DeForest, WI (US)

(73) Assignee: ABS Global, Inc., DeForest, WI (US)

(21) Appl. No.: 18/050,215

(22) Filed: Oct. 27, 2022

(51) Int. Cl. C12N 5/071; C12N 5/00

(52) U.S. Cl. CPC C12N 5/0612; C12N 5/0081

(57) ABSTRACT

The present teachings provide for methods of separating live and dead spermatozoa cells from non-human mammalian sex-selected semen. Sex-selected semen is first treated with proteinase K and TCEP, then applied to a density gradient media for separation. Separation is typically accomplished through centrifugation. Commercially available density gradient media include PERCOLL® and BOVIPURE®. Separation of live and dead cells in sex selected semen allows for precise quantification of sex skew using ddPCR™.

### FIELD OF DISCLOSURE

Technologies for assessing, quantifying, and isolating sperm cell populations and/or subpopulations are provided, as well as methods and systems to assess the efficacy of semen sexing methods.

### BACKGROUND

High purity sperm cell populations that have been differentiated based on chromosomal differences—such as, for example, sperm cell populations that are skewed toward X-chromosome bearing or Y-chromosome bearing populations of spermatozoa, rather than the naturally-occurring 50:50 X:Y chromosome split—can be utilized to accomplish in vitro or in vivo fertilization, including artificial insemination (AI) or in vitro fertilization (IVF) of ova or oocytes of numerous mammals such as bovids, equids, ovids, goats, swine, dogs, cats, camels, elephants, or the like. See, e.g., U.S. Pat. No. 5,135,759.

Quantifying the relative population of sperm cells bearing the X or Y chromosome in a sexed semen sample has historically been limited to methods that are either low throughput and sensitive to user subjectivity (like fluorescent in situ hybridization), or relatively insensitive (like qPCR with a change detection threshold of 2×). Customers pay a premium for sexed semen, and therefore desire to have access to reliable quality control data, which include an accurate, precise test for sex skew that is orthogonal to the method used to generate sexed semen.

However, conventional technologies are significantly limited in their ability to reliably assess the degree to which populations have been effectively skewed to X-chromosome bearing and Y-chromosome bearing populations. This can result in spermatozoa populations having unrecognized underrepresentation of the population of interest, or unrecognized overrepresentation of the population that is not of interest.

Sexed semen from sires with desired genetic traits provides farmers with the opportunity to advance herd genetics, and therefore farm profitability, while ensuring calves predominately of the desired sex are born as the herd turns over. Dairy farmers, for example, utilize X-skewed sexed semen (enriched for X chromosome sperm) to maintain a female, milk-producing herd. The ultimate utility of sexed semen is dependent upon the relative percentage of X or Y chromosome bearing sperm (‘female or male cells’) present in the product, and therefore dependent upon the accuracy of both making sexed semen and verifying the final sex skew.

Commercial sexed semen is currently produced by staining sperm with Hoechst 33342, a dye that penetrates live cells, binds stoichiometrically to DNA, and releases a fluorescent signal which quantitatively reflects total cellular DNA content when binding has been driven to completion. Custom, high-throughput sexing cytometers discriminate the roughly 4% difference in total DNA content between X and Y chromosome containing sperm (due to the relative size of the sex chromosomes) by quantifying Hoechst 33342 emission fluorescence. Once the sex of the sperm is determined, sperm are either segregated into separate containers based on sex, or the undesired cell population is eliminated by laser ablation. The maximum achievable sex skew (enrichment for either the X or Y population) is at minimum dependent upon how well the cells are stained, alignment of the pancake shaped sperm head in the optical detection plane, and speed at which sperm flow through the cytometers.

Given the inherent challenges in separating two cell populations based on a 4% difference in total DNA content (Seidel, G. E., Animal, 8 Suppl 1:160-164 (2014)), the resulting product is variable in its final sex skew. The percentage of female calves born from the thousands of sexed semen inseminations most likely reflects variance in the actual skew of sexed semen straws.

An accurate, precise method for quantifying the sex skew of unfrozen sexed semen or of frozen-thawed sexed semen is critical to define the current sexed semen products for farmers, and subsequently to improve the skew to meet market demands. Many existing approaches have drawbacks that limit their usefulness for robustly quantifying the sex-skew of sexed semen. Available orthogonal assays (FISH and qPCR) specifically identify X and Y chromosomes, but lack quantitative sensitivity. Using semen sexing cytometers to quantify sex skew utilizes the same DNA binding dye and detection scheme that was originally used to identify X or Y chromosome containing sperm during the sexing process, and is therefore subject to the same variations or biases that may have been present when producing the sex skewed product. Such pitfalls may include resolution limits imposed by flow speed, suboptimal cell orientation during fluorescence quantification, or DNA staining efficiency. This approach fails to confirm product quality with an independent, orthogonal test, and does not use unique, positive identifiers for either the X or Y chromosome.

Unlike other techniques, droplet digital PCR (ddPCR) provides an accurate and precise sex skew measurement by subdividing a pool of template DNA into nanoliter scale droplets containing either one or zero copies of template DNA. PCR amplification occurs in these droplets, and the number of copies of the amplicon of interest can be counted as the number of fluorescence-positive droplets based on classic qPCR fluorescent reporters. Methods and systems for optimized and validated multiplexed ddPCR assays that use a copy counting method to quantify the sex skew (ratio of X or Y chromosomes) in frozen-thawed bovine sexed semen, for copy counting in sex selected semen prior to the freezing process, and for validating additional primers for use in ddPCR are provided in U.S. Pat. No. 10,961,577B2, entitled METHODS AND SYSTEMS FOR ASSESSING AND/OR QUANTIFYING SPERM CELL SUBPOPULATIONS BEARING A SPECIFIC GENETIC SIGNATURE, issued 30 Mar. 2021, by Elon Roti-Roti, et al., which is incorporated by reference herein in its entirety.

However, because ddPCR is extremely sensitive, the presence of dead cells in the preparation can lead to misleading PCR results, as DNA from dead cells may still be amplified if the dead cells are not separated prior to PCR, thus not reflecting the skew of living cells that can produce a pregnancy. The current gold standard method of separating dead cells from the live cells includes the use glass wool, which is expensive, produces results that are hard to reproduce, and requires the use of a fume hood. Safer, more consistent methods of separating live cells from dead cells are needed.

### SUMMARY OF THE INVENTION

The present teachings provide a method of separating live cells from dead cells in sex selected semen by treating the semen with a mixture of proteinase K and TCEP, then separating the cells using a density gradient media that is safe to use on a benchtop.

In one embodiment, what is provided is a method of removing dead cells from sex selected semen for DNA extraction. The method comprises treating non-human mammalian sex selected semen with proteinase K and tris(2-carboxyethyl)phosphine (TCEP) to form treated semen and separating dead cells in the treated semen from live cells in the treated semen using a density gradient media.

In various embodiments the density gradient media is polyvinylpyrrolidone (PVP) coated colloidal silica.

In various embodiments the density gradient media is silane coated colloidal silica.

In various embodiments the separating comprises layering the treated semen onto the density gradient media to form a layered sample and then centrifuging the layered sample.

In various embodiments the non-human mammalian sex selected semen is treated with 0.1-1.5 U/ml proteinase K.

In various embodiments the non-human mammalian sex selected semen is treated with 0.23-1.20.9 U/ml proteinase K.

In various embodiments the non-human mammalian sex selected semen is treated with about 0.75 U/ml proteinase K.

In various embodiments the non-human mammalian sex selected semen is treated with 1 mM-10 mM TCEP.

In various embodiments the non-human mammalian sex selected semen is treated with 5-10 mM TCEP.

In various embodiments the non-human mammalian sex selected semen is treated with about 7.5 mM TCEP.

In various embodiments the density gradient media is an 85% solution of polyvinylpyrrolidone (PVP) coated colloidal silica.

In various embodiments the non-human mammalian sex selected semen is a frozen-thawed sample.

In one embodiment, what is provided is a method of removing dead cells for assessing skew on sex selected non-human mammalian semen. The method comprises: treating non-human mammalian sex selected semen with proteinase K and TCEP to form treated semen; depositing the treated semen onto a density gradient media to form a layered sample; centrifuging the layered sample to form a supernatant and pellet; aspirating the supernatant; washing the density gradient media out of the pellet; extracting DNA from the pellet; and performing digital droplet PCR to assess sex skew.

In various embodiments the density gradient media is an 85% solution of polyvinylpyrrolidone (PVP) coated colloidal silica.

In various embodiments the non-human mammalian sex selected semen is treated with 0.1-1 U/ml proteinase K.

In various embodiments the non-human mammalian sex selected semen is treated with 0.3-0.9 U/ml proteinase K.

In various embodiments the non-human mammalian sex-selected semen is treated with about 0.75 U/ml of proteinase K.

In various embodiments the non-human mammalian sex selected semen is treated with 1 mM-10 mM TCEP.

In various embodiments the non-human mammalian sex selected semen is treated with 4-9 mM TCEP.

In various embodiments the non-human mammalian sex selected semen is treated with about 7.5 mM TCEP.

In various embodiments the non-human mammalian sex selected semen is treated with about 0.5 U/ml of proteinase K and about 7.5 mM TCEP; the density gradient media is an 85% solution of polyvinylpyrrolidone (PVP) coated colloidal silica; and the centrifuging is at 1000 RCF for 30 minutes.

In various embodiments the non-human mammalian sex selected semen is a frozen-thawed sample.

In another embodiment, what is provided is a method of assessing a sex skew of sex selected non-human mammalian semen comprising: treating non-human mammalian sex selected semen with about 0.5 U/ml of proteinase K and 7.5 mM TCEP to form treated semen; depositing the treated semen onto an 85% solution of polyvinylpyrrolidone (PVP) coated colloidal silica to form a layered sample; centrifuging the layered sample at 1000 RCF for 30 minutes to form a supernatant and pellet; aspirating the supernatant; washing the colloidal silica coated with PVP out of the pellet; extracting DNA from the pellet; and performing digital droplet PCR to assess sex skew.

Further embodiments of the invention are disclosed throughout other areas of the specification and claims.

### BRIEF DESCRIPTION OF THE DRAWINGS

FIG. 1 provides a box-plot graph illustrating X-skew of a treatment of an embodiment of the present teachings to sexed semen samples compared to glass wool over 18 samples.

FIG. 2 provides a box-plot graph illustrating X-skew of treatments of embodiments of the present teachings to sexed semen samples compared to glass wool in two different cattle breeds (Bos indicus and water buffalo).

FIG. 3 provides a box-plot graph illustrating consistency of delta skew between users of methods of embodiments of the present teachings.

FIG. 4 provides a box-plot graph illustrating consistency of skew between glass wool and a method of the present teachings, even when x-skew is low.

The examples set out herein illustrate several embodiments of the present disclosure but should not be construed as limiting the scope of the present disclosure in any manner.

### DETAILED DESCRIPTION

In general, the present technology encompasses a method of removing dead cells from a sex selected semen sample to prepare the sex selected semen sample for DNA extraction. In some embodiments, a method of the present teachings can comprise treating non-human mammalian sex selected semen with proteinase K and tris(2-carboxyethyl)phosphine (TCEP) to form treated semen; and separating dead cells in the treated semen from live cells in the treated semen using a density gradient media. Density gradient media of the present teachings is safe to use on a bench top. The present method also requires less input for measuring of sex skew and is more cost effective than previous methods.

Without being limited by theory, TCEP is a reducing agent that breaks the chemical bonds between protamine and DNA. TCEP can be obtained from a variety of commercial vendors known to skilled artisans such as UBP Bio, ThermoFisher, and Millipore Sigma (formerly Sigma Aldrich). Proteinase K is a serine proteinase with a broad spectrum of activity originally isolated from Engyodontium album, which aids in breaking down the protamine discs released by TCEP; proteinase K also breaks down other proteins accessible in the dead sperm cells. Proteinase K can be obtained from a variety of commercial vendors including Millipore Sigma, ThermoFisher, InVitrogen, and New England Biolabs. Together, these treatments decrease the density of dead sperm cells, allowing them to be separated using a density gradient media.

As used herein, a density gradient media can be a non-toxic medium that is more dense than the TCEP/proteinase K treated dead cells, but less dense than the live cells. This density differential can cause the dead cells to float to the top and the live cells to settle in the bottom. This separation can be enhanced by spinning the sample in a centrifuge (“centrifuging”); the live cells will form a pellet in the bottom of the gradient while the dead cells will remain the supernatant.

Non-limiting examples of density gradient media include colloidal silica, polyvinylpyrrolidone (PVP) coated colloidal silica, silane coated colloidal silica, and sucrose. PVP coated colloidal silica is sold under the name PERCOLL® (CYTIVA®, Marlborough, MA). Silane coated colloidal silica is sold under the trade names PERCOLL® Plus and BOVIPURE™ (Nidacon Laboratories, Molndal, Sweden). An exemplary sucrose based density gradient medium includes 30% sucrose dissolved in water. (Sucrose is commercially available from most chemical vendors.)

In general, in performing a method of the present teachings, a skilled artisan treats sex selected semen with 0.1-1 U/ml of proteinase K and 1 mM to 10 mM TCEP to form treated semen. The treated semen is then contacted with a density gradient medium to separate the live cells from the dead cells. The live cells are collected and DNA is extracted through any means known in the art, including any of a large number of kits, for example (but without limitation), the PureLink™ DNA Extraction Kit (Kit brand: Invitrogen, Trademark owner: Thermo Fisher Scientific, Waltham, MA), DNEASY® Blood & Tissue Kit (QIAGEN® Group, Hilden, Germany), MAXWELL® CSC Genomic DNA Kit (PROMEGA®, Madison, WI), GenElute™ Mammalian Genomic DNA Isolation Kit (MilliporeSigma, St. Louis, MO), or any other suitable DNA preparation kit known to skilled artisans.

In various embodiments, the sex selected semen is treated with 0.1-1 U/ml of proteinase K. In various configurations, the sex selected semen is treated with 0.3-0.9 U/ml of proteinase K. The sex selected treatment can be treated with about 0.1, about 0.2, about 0.3, about 0.4, about 0.5, about 0.6, about 0.7, about 0.8, about 0.9, or about 1.0 U/ml of proteinase K. In a preferred embodiment, the sex selected semen can be treated with about 0.75 U/ml of proteinase K.

In various embodiments, the sex selected semen can be treated with 1 mM to 10 mM TCEP. In some configurations, the sex selected semen can be treated with 4-9 mM TCEP. In a preferred configuration, the sex selected treatment can be treated with 7.5 mM TCEP. The sex selected semen can be treated with about 1, about 2, about 3, about 4, about 5, about 6, about 7, about 8, about 9, or about 10 mM TCEP. Treatment with TCEP alone is possible, but the use of proteinase K increases cell yields.

The timing of the proteinase K/TCEP treatment can be such that the reagents have enough time to digest the dead cells, however, longer incubations lead to diminishing returns as overdigestion will lead to destruction of live cells. The treatment can be from 5-20 minutes, with an ideal time of about 15 minutes. The treatment can be for 5, 6, 7, 8, 9, 10, 11, 12, 13, 14, 15, 16, 17, 18, 19, or 20 minutes.

The concentrations of density gradient media will vary according to the density gradient media used. For example, for pvp coated colloidal silica (PERCOLL®), the concentration can be from 75% to 90% PVP coated colloidal silica in a single gradient, that allows for a self-assembling gradient. The concentration can be about 75%, about 76%, about 77%, about 78%, about 79%, about 80%, about 81%, about 82%, about 83%, about 84%, about 85%, about 86%, about 87%, about 88%, about 89%, or about 90%. In a preferred configuration, the concentration of coated colloidal silica can be about 85%. Alternatively, or in addition, the density gradient media can be a two concentration gradient of 45% PVP coated colloidal silica layered onto 90% PVP coated colloidal silica.

When silane coated colloidal silica (BOVIPURE™) is used for a self-assembling gradient, the concentration may range from 35%-65% silane coated colloidal silica. In various configurations, the concentration may range from 40%-60% silane coated colloidal silica. The concentration be about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60% silane coated colloidal silica. Alternatively, or in addition, a gradient may be pre-established with about 40% silane coated colloidal silica and about 80% silane coated colloidal silica.

When sucrose is used, a self-assembling gradient of 25%-50% sucrose may be used. In various configurations, a self-assembling gradient of 30-40% sucrose can be used. The self-assembling gradient can be about 40%, about 41%, about 42%, about 43%, about 44%, about 45%, about 46%, about 47%, about 48%, about 49%, or about 50% sucrose.

Various methods of using density gradient media to separate live from dead cells are known in the art. These methods include swim-up and centrifugation. The sperm swim-up procedure takes advantage of the natural gravitropic of live sperm—the semen is placed at the bottom of the gradient and live sperm are allowed to “swim-up” to the top of the gradient. This technique is suitable for non-time sensitive applications.

Alternatively, or in addition, the dead and live sperm cells can be separated via centrifugation. For this application, the density gradient medium can be placed into a centrifuge tube or other container suitable for centrifugation. In general, the treated semen can be deposited onto the gradient. This can be accomplished by slow pipetting onto the top of the density gradient media, to form a layer of treated sperm on top of the density gradient medium.

This dual layer composition is then centrifuged. The precise speed is dependent on the density gradient medium and can be slow for a long period of time—for example 300×g for 30 minutes, or a short period of time at larger speed, for example 10,000×g for 3 minutes, or anywhere in between in a period of time that allows the live cells to pellet without killing them. A preferred configuration is to centrifuge at approximately 1000×g for 30 minutes. The soft ramp, slow breaking, or slow ramp setting can be used.

The methods described above are particularly useful for clean up of sex selected semen prior to digital droplet PCR (ddPCR) for determination of sex skew and sample quality. Methods of sex selecting semen and ddPCR are discussed below.

### Differentiation Technologies

A number of techniques, directly or indirectly based on differences in size, mass, or density have been disclosed for use in discriminating X-chromosome-bearing from Y-chromosome-bearing spermatozoa. The most commonly used methods utilize flow cytometer techniques for the sex-skewing of spermatozoa and generally involve staining spermatozoa with a fluorochrome; the stained spermatozoa are made to flow in a narrow stream or band passing by an excitation or irradiation source such as a laser beam. As stained particles or cells pass through the excitation or irradiation source, the fluorochrome emits fluorescent light. An optical lens assembly collects the fluorescent light, focused on a detector—typically a photomultiplier tube—that generates and multiplies an electronic signal, which may then be analyzed by an analyzer. The data can then be displayed as multiple or single parameter chromatograms or histograms. The number of cells and fluorescence per cell may be used as coordinates. Detection of the two populations provides the opportunity to skew the population towards one population or the other, including by sorting X- and Y-chromosome bearing cells into separate populations, enriching the population for either X- or Y-chromosome bearing cells, or selectively removing, destroying, or otherwise inactivating either X- or Y-chromosome bearing cells in a population. However, with respect to this type of technology a variety of problems remain unresolved, and ensuring that chromosomal differentiation techniques yield highly purified populations (e.g. X-chromosome bearing or Y-chromosome bearing sperm cells) can be difficult.

In the following description and examples, a number of terms are used. In order to provide a clear and consistent understanding of the description and claims, including the scope to be given such terms, definitions are provided for the terms as used in the description and claims. Unless otherwise defined herein, all technical and scientific terms used have the same meaning as commonly understood by one of ordinary skill in the art to which this invention belongs.

The present disclosure aids in the determination of the efficacy of techniques for differentiating sperm cells based on chromosomal differences by removing dead sperm cells. Such determination is essential for ensuring the quality of the sex-selected cells, and the success of subsequent use of those cells, for example in production of offspring having characteristics based on those chromosomal differences. In one aspect, sperm cells may be sex-selected based on the presence of an X-chromosome or a Y-chromosome, and the quality of that differentiation must be accurately assessed so as to permit reliable sex-selection of offspring. Sperm cells may also be differentiated based on other chromosomal differences, and confirmation of that differentiation must also be reliably made. One useful method of determining skew is the use of digital droplet PCR (ddPCR) which is described in detail in U.S. Pat. No. 10,961,577 by Roti Roti, et al.

In one aspect, the present disclosure relates to chromosomal discrimination or differentiation of sperm cells. Because sperm cells are haploid and contain only a single copy of each chromosome, individual sperm cells can be differentiated based on which copies of the chromosomes they possess. As used herein a “differentiation region” refers to a chromosome, or a specific portion or combination of portions thereof (e.g. an allele or haplotype) that permits differentiation of individual sperm cells in a population. For example, each sperm cell in a population obtained from a single male animal will contain one of two copies of a particular gene, which may be represented by two different alleles in the animal (i.e. the animal is heterozygous at that locus). Therefore, sperm cells in the population obtained from that animal can be differentiated based on which copy (or allele) they possess. Alternatively, sperm cells possess either a X chromosome or a Y chromosome, and can be differentiated based on which chromosome they possess. Chromosomal differentiation also includes selective identification or recognition of chromosomal DNA, including by binding to or otherwise recognizing a differentiation region. For example, this includes, but is not limited to, the binding of oligonucleotides to specific target chromosomal DNA sequences, such as antisense primers or guide RNAs. Discrimination of the sperm cells can be achieved using a variety of techniques, including but not limited to flow cytometry, magnetic techniques, columnar techniques, gravimetric techniques, use of electrical properties, combinations of electrical and gravimetric properties, use of motility properties, chemical techniques involving monoclonal antibodies and/or membrane proteins. Chromosomal differentiation may include sex skewing.

Sex skewing as used herein refers to altering a population of sperm cells to increase or decrease the proportion of X-chromosome or Y-chromosome bearing sperm cells. Methods for sex skewing include, but are not limited to, flow cytometric differentiation using the quantitative difference in the DNA content of X and Y chromosomes and consequently in the DNA content of X- and Y-bearing sperm. Other methods for differentiating using the quantitative difference in the DNA content of X and Y chromosomes and consequently in the DNA content of X- and Y-bearing sperm, without using dyes, are described in U.S. Pat. No. 9,683,922. U.S. Pat. No. 5,135,759 issued to Johnson involves individual discrimination and separation of the sperm through the techniques of flow cytometry; magnetic techniques, such as appears disclosed in U.S. Pat. No. 4,276,139, to columnar techniques as appears disclosed in U.S. Pat. No. 5,514,537, to gravimetric techniques as discussed in U.S. Pat. No. 3,894,529, U.S. Reissue Patent No. 32350, U.S. Pat. Nos. 4,092,229, 4,067,965, and 4,155,831. Use of electrical properties has also been attempted as shown in U.S. Pat. No. 4,083,957 as well as a combination of electrical and gravimetric properties as discussed in U.S. Pat. Nos. 4,225,405, 4,698,142, and 4,749,458. Use of motility properties has also been attempted as shown in U.S. Pat. Nos. 4,009,260 and 4,339,434. Chemical techniques have also been disclosed, such as those shown in U.S. Pat. Nos. 4,511,661 and 4,999,283 (involving monoclonal antibodies) and U.S. Pat. Nos. 5,021,244, 5,346,990, 5,439,362, and 5,660,997 (involving membrane proteins), and U.S. Pat. Nos. 3,687,803, 4,191,749, 4,448,767, and 4,680,258 (involving antibodies) as well as the addition of serum components as shown in U.S. Pat. No. 4,085,205. The production of chromosomally differentiated sperm cell populations, including sex-skewed (e.g. X-skewed) sperm cells can be achieved using a variety of approaches, including flow cytometric sorting or separation (e.g. by droplet sorting) or using focused energy, for example to inactivate certain differentiated sperm cells, as described in U.S. Pat. No. 9,588,100.

Differentiated sperm cell populations can be provided in a variety of forms. Differentiated sperm cell populations may be frozen or unfrozen, and may be in media that include one or more of water, salts (e.g. NaCl, KCl, Na₂HPO₄, NaHCO₃, MgCl₂·6H₂O), glucose, sodium pyruvate, sodium lactate, HEPES, bovine serum albumin (BSA), sodium hydroxide, tris, extenders (e.g. egg yolk), cryo-preservatives (e.g. glycerol), dyes, and antibiotics. Samples may also include compounds that inactivate or decrease cell motility, or increase viability. Inactivated or dead cells, or cell components or debris, may also be present in differentiated samples. In an exemplary embodiment, the sperm cell population comprises primarily X-chromosome bearing sperm in the presence of dead or inactivated sperm cells and cell debris, wherein the sperm cells are in a cryopreservative-containing media. In another exemplary embodiment, the sperm cell population comprises a separated sperm cell population that is primarily X-chromosome bearing sperm cells. The present disclosure provides methods of removing the dead or inactivated sperm cells and cell debris, to allow for more accurate assessment of skew using ddPCR or similar molecular techniques.

Providing sperm cell samples with an accurately assessed population enrichment, in accordance with the invention, can be achieved with the sperm cells obtained from numerous and varied mammals, including without limitation, bovines, equines, ovines, canines, felines, swine, marine mammals such as cetaceans and pinnipeds, Cervidae such as deer and elk, or primates. Preferred mammals include ungulates such as bovines, swines, and ovines. Especially preferred mammals include livestock species such as cattle, pigs, sheep, horses, and goats.

The term primer refers to a single-stranded oligonucleotide capable of acting as a point of initiation of template-directed DNA synthesis under appropriate conditions (i.e., in the presence of four different nucleotide triphosphates and an agent for polymerization, such as, DNA or RNA polymerase or reverse transcriptase) in an appropriate buffer and at a suitable temperature. The appropriate length of a primer depends on the intended use of the primer but typically ranges from 15 to 30 nucleotides. Short primer molecules generally require cooler temperatures to form sufficiently stable hybrid complexes with the template. A primer need not reflect the exact sequence of the template but must be sufficiently complementary to hybridize with the template. The term primer site, or priming site, refers to the area of the target DNA to which a primer hybridizes. The term primer pair means a set of primers including a 5′ upstream primer that hybridizes with the 5′ end of the DNA sequence to be amplified and a 3′ downstream primer that hybridizes with the complement of the 3′ end of the sequence to be amplified.

High purity separated spermatozoa from the various species of mammals can be incorporated into products that can be used with artificial insemination protocols or as part of commercial business methods such as those as described in U.S. Provisional Application Nos. 60/211,093, 60/224,050, or International Application No. PCT/US1999/017165; or be used with low dose insemination protocols as described in International Application No. PCT/US1998/027909, or used for in vitro fertilization of oocytes from animals, including humans, as described in U.S. Provisional Application No. 60/253,785 and U.S. patent application Ser. No. 15/476,509, each of the above-mentioned references are hereby incorporated by reference.

### Assessment of Chromosomal Differentiation

In one aspect, the present disclosure provides systems and methods for accurate, quantitative assessment of chromosomal differentiation of sperm cells having either X- or Y-chromosomes (e.g., sex-skewed sperm cells, semen, or extended semen). Assessment of sex skew generally involves a determination of the overall representation of a particular chromosome, or a portion thereof, in a sperm cell population. For example, in a sperm cell population that has been sex-selected, assessment of sex skew will determine the representation of the X chromosome and the Y chromosome in the population as a whole, thereby quantifying the sex-skew. Sperm cell samples subjected to sex selection can be assessed using these systems and methods, providing information and data about the sperm cell population that is important for subsequent applications, including, for example, use of the sperm cells in fertilization. In a further aspect, the sperm cell population can be assessed for the presence or absence of one or more particular chromosomes, or a particular region or sequence of a chromosome, and the amount of each chromosome, or region or sequence thereof, can be quantified with respect to a housekeeping or reference gene.

Depending on the application, amplification may be useful in achieving desired goals in the detection of chromosomal differentiation region nucleic acid sequences. For example, the Y specific sequences of chromosomal DNA from a sperm cell population can be highly amplified.

A PCR method better suited to large scale assay processing is an automated programmable system. One such system has been developed, as described in Weier, H. U., Gray, J. W., DNA, 1988, 7, pp. 441-447. This article also describes the use of a thermostable DNA polymerase which is particularly suited to the production of the subject DNA probe and to the amplification of the test sample material. More advanced automated programmable systems especially suited for the present disclosure include the Droplet Digital PCR (ddPCR™) produced by BioRad. ddPCR is an adaptation of qPCR or realtime PCR in which a fluorescent probe reports amplification, but the reaction is divided into thousands of droplets. The ddPCR technique is described by Hindson, B. J., et al., Anal. Chem., 2011, 83, 8604-8610. The template for ddPCR is optimally distributed so that each droplet contains one copy of the target template, and as a result the readout is counting the number of fluorescent-positive droplets. As with other PCR techniques, ddPCR requires a source of DNA, such as for example isolated genomic DNA. ddPCR is a PCR method that is based on water-oil emulsion droplet technology, wherein each sample is fractionated into around 20,000 or more droplets, and PCR amplification of the template DNA occurs in each individual droplet. ddPCR technology uses reagents and workflows similar to those used for most standard TaqMan probe-based assays. The massive sample partitioning is a key aspect of the ddPCR technique, allowing for simultaneous multiplication of amplification (and detection), up to 1000-fold over other PCR techniques. The PCR reaction can be carried out with one or more primer pairs, to amplify one or more corresponding regions in the template DNA. The PCR reaction can be carried out in the presence of one or more probes, which are specific for and allow for specific detection of the one or more amplified regions. Following PCR, each droplet is analyzed or read in a flow cytometer to determine the fraction of droplets in which the target DNA was amplified, typically by detecting fluorescence emitted by the specific probes.

An exemplary sex skew assay is designed to interrogate one target each on the X and Y chromosomes as well as an autosomal target to confirm total cells counted. Optimizing a triplex ddPCR assay requires maximizing separation of the droplet populations containing only individual target amplicons (X, Y, or GAPDH), as well as the dual and triple occupancy droplets (X+Y, X+GAPDH, Y+GAPDH, and X+Y+GAPDH), which can be challenging when utilizing a 2-channel detection platform. Two amplicons can be discretely identified, one in each of the two fluorescent channels utilizing HEX and FAM probes for the two targets, respectively. The third amplicon is detected using a mix of two probes which are sequence identical, but differentially tagged with either HEX or FAM fluorophores. A robust sex skew assay intended as a quality control metric also requires assaying as many cells as necessary from a single semen straw without compromising the ability to separate the target populations. To achieve the necessary quality control target for validation, input quantities for template genomic DNA and each primer and probe are iteratively adjusted until maximal separation of the fluorescent droplet populations is achieved, while assaying the maximal total number of cells. The methods of the present teachings allow for higher quality input template DNA, as the removal of undesirable cells.

A 1:1 autosomal:(X+Y) copy ratio is observed in conventional semen samples obtained from genomic DNA isolated from the viable sperm population in frozen-thawed sex skewed semen straws. However, given the unique nature of some sex skewed semen samples, such as an X skewed sexed semen sample obtained by the laser ablation of Y chromosome sperm, which contains laser-sliced cells predominantly comprising Y chromosome sperm, attention is needed to clean up the samples prior to genomic DNA extraction. If DNA is extracted from a sexed semen sample, whether the extraction occurs immediately after a sexing or skewing process or after a freeze-thaw processes, without removing the laser-killed cells, the expected result is an apparent 50/50 X/Y chromosome representation. This does not, however, reflect the viable, motile population which provides the cells that will ultimately produce a pregnancy. The present specification provides for removing non-viable sperm from the sample prior to genomic DNA extraction.

An objective of the present disclosure is assessing or validating chromosomal differentiation, that is, to determine whether the chromosomes present in a differentiated sample in fact comprise those that have been selected for.

### Use of Differentiated Sperm Cells for Fertilization

The ultimate goal of the present disclosure is to provide high quality, sex-selected semen for the fertilization of an egg or insemination of a female mammal, generally employing the novel process for sorting spermatozoa as described above. Once a sex-selected sperm cell population has been assessed as discussed in greater detail above, the sperm cell population may be used to inseminate a female mammal. Insemination may be performed according to any of a number of methods well known to those of skill in the art. These methods include, for example, artificial insemination, including standard artificial insemination and deep uterine insemination, and other methods well known to those of skill in the art. Alternatively, the sex-selected semen may be used to fertilize an egg using in vitro fertilization, intracytoplasmic sperm injection (ICSI), and other methods well known to those in the art. The fertilized egg may thereafter be introduced into the uterus of a female mammal by any of a number of means well known to those of skill in the art, such as for example embryo transplant.

The following examples are offered by way of illustration only and should not be construed as intending to limit the claimed technology in any manner.

### Example 1

This example illustrates preparation of reagents for use in a method of the present teachings.

10×PERCOLL® salt solution was made by dissolving 8.76 g of NaCl, 2 ml of 1M Ca₂Cl, and 4 ml of 100 mM MgCl₂ in a total of 100 ml of water (final concentrations: 150 mM NaCl, 20 mM Ca₂Cl, and 4 mM MgCl₂). This solution was filter sterilized and stored at 4° C. until ready for use. 85% Percoll® (PVP coated colloidal silica) was made by adding 5 ml of 10×PERCOLL® salt solution, 2.5 ml of water, and 42.5 ml of PERCOLLx and stored at 4° C. until ready for use.

TCEP/Proteinase K digestion buffer was made on the day of sample preparation as follows:

| Reagents | 6 batches | | 12 batches | | 24 batches | |
|---|---|---|---|---|---|---|
| 10X Tris | 225 | μL | 450 | μL | 900 | μL |
| Milli-Q Water | 2 | mL | 4 | mL | 8 | mL |
| 1M TCEP | 16.9 | μL | 33.8 | μL | 67.6 | μL |
| Invitrogen 20 mg/mL | 4.5 | μL | 9 | μL | 18 | μL |
| (>20 U/mg) | | | | | | |
| Proteinase K | | | | | | |

### Example 2

This example illustrates treatment of batches of frozen sexed semen with TCEP and proteinase K.

One straw per sex selection batch was thawed by placing each straw in the straw thawer for 45 seconds. After 45 seconds, each straw was removed and dried off; the sealed end was cut, and then the straw was plunged into a 1.7 ml tube. 1 mL of room temperature 1×Tris buffer was added, and the tube was invert several times to homogenize the 1×Tris and straw contents. The tubes were then centrifuged for 10 minutes at 300 rcf. The 1×Tris/extender mixture was aspirated off of each of the pellets, leaving <5011.1 after aspiration. 30011.1 of the treatment buffer (see Example 1) was then added to each of the cell pellets. Each pellet was vortexed to resuspend, and then the tubes were incubated with rotation at 37° C. for 15 minutes.

### Example 3

This example illustrates treatment of fresh sexed semen with TCEP and proteinase K.

5 ml samples were removed from the output of a sex selection instrument and 1 mL of room temperature 1×Tris buffer was added. The tube was inverted several times to homogenize the 1×Tris and collected throughput. The tubes were then centrifuged for 10 minutes at 300 rcf. The 1×Tris/sex selection buffer mixture was aspirated off of each of the pellets, leaving <5011.1 after aspiration. 30011.1 of the treatment buffer (see Example 1) was then added to each of the cell pellets. Each pellet was vortexed to resuspend, and then the tubes were incubated with rotation at 37° C. for 15 minutes.

### Example 4

This example illustrates separation of live cells from cellular debris using PERCOLL® as the density gradient media.

The TCEP/Proteinase K treated samples from Example 2 or Example 3 were pulse spun and then deposited on top of an 85% Percoll single layer in a tube. These tubes were then spun in a centrifuge for 30 minutes at 1000 rcf with the soft ramp setting on. The supernatant was aspirated from the top down, until less than 50 μl remained. The pellet was then resuspended in 75 μL of Stain TALP that was removed from a tube containing 1 ml of Stain TALP. The resuspended pellet was then transferred to the tube of Stain TALP. The tube was then spun at 300 rcf for 10 minutes. The supernatant was aspirated from the top down, until less than 5011.1 remained. 1 mL room temperature Stain TALP was added to each pellet. The tubes were again spun at 300 rcf for 10-minutes.

The supernatant was then aspirated to about 200 μl and the tubes vigorously vortexed. The DNA was then extracted using the PureLink™ DNA Extraction Kit according to the manufacturer's directions.

### Example 5

This example illustrates assessing the quality of sex-skewing of sperm cells.

Digital droplet PCR (ddPCR™) is an adaptation of quantitative PCR (qPCR) or realtime PCR in which a fluorescent probe reports amplification, but the reaction is divided into thousands of droplets. Template DNA for use in ddPCR™ is optimally distributed so each droplet contains one copy of the target template, so the readout is counting the number of fluorescent-positive droplets.

There are essentially five main steps involved in the ddPCR™ assay workflow: Genomic DNA isolation, droplet generation; thermocycling; droplet reading; and data analysis.

The present inventors use an in-house developed ddPCR™ assay that verifies the cells counted per reaction using a housekeeping gene, an independent assay of the X and Y chromosomes in a method that allows quantification of at least 2,000 cells across duplicate reaction wells, and with less than 1% variance in the percentage of X chromosome bearing cells detected in replicate reads.

The assay is presented in U.S. Pat. No. 10,961,577B2. DNA was extracted from sex selected semen as described in Example 2-Example 4. Concentration and purity of DNA samples was measured and the DNA was diluted to 2.88 ng/μL.

ddPCR™ was carried out on the samples using the BIO-RAD® DROPLET DIGITAL™ PCR (ddPCR™; BIO-RAD® Laboratories, Inc., Hercules, CA) system, according to manufacturer's instructions. Droplets are generated using the QX200 Droplet Generator and analysis was carried out using the QX200 Droplet Reader, in the presence of the primers and probes directed to the X chromosome, Y chromosome, and the GAPDH gene for normalization.

### Example 6

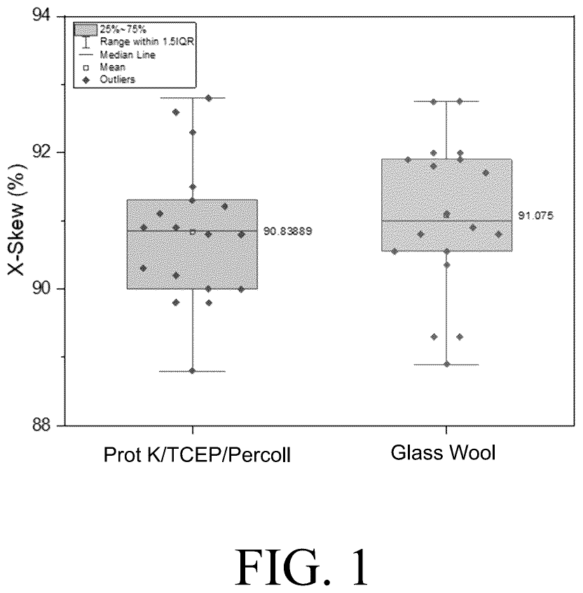

This example illustrates a comparison of x-skew between the gold standard glass wool treatment (see, e.g., U.S. Pat. No. 5,575,914) and a technique of the present teachings. Eighteen samples were sex selected and then split into two samples for comparative processing. The samples were run by two different users. The control sample was run through a glass wool column (0.03 g in a 1 cc syringe packed to a 0.1 mL volume) by gravity drip (112X-475 borosilicate, Johns Manville, Denver, CO) to remove laser-killed cells. The treatment sample was treated with TCEP and Proteinase K as described in Example 2 and then run on a PERCOLL® gradient as described in Example 4. X-skew was then measured using ddPCR™ according to Example 5. The results are shown in FIG. 1. This example illustrates that the present teachings produce x-skew comparable to that of glass wool.

### Example 7

This example illustrates a comparison of x-skew between the gold standard glass wool treatment (see, e.g., U.S. Pat. No. 5,575,914) and a technique of the present teachings in different cattle breeds.

Ejaculates from both the Zebu variety of Bos indicusand from water buffalo were sex selected and then split into two samples for comparative processing. A sample size of 40 batches was used for Zebu and a sample size of 20 batches was used for water buffalo. The control sample was run through a glass wool column (0.03 g in a 1 cc syringe packed to a 0.1 mL volume) by gravity drip (112X-475 borosilicate, Johns Manville, Denver, CO) to remove laser-killed cells. The treatment sample was treated with TCEP and Proteinase K as described in Example 2 and then run on a PERCOLL® gradient as described in Example 4. X-skew was then measured using ddPCR according to Example 5. The results are shown in FIG. 1 provides a box-plot graph illustrating X-skew of a treatment of an embodiment of the present teachings to sexed semen samples compared to glass wool over 18 samples.

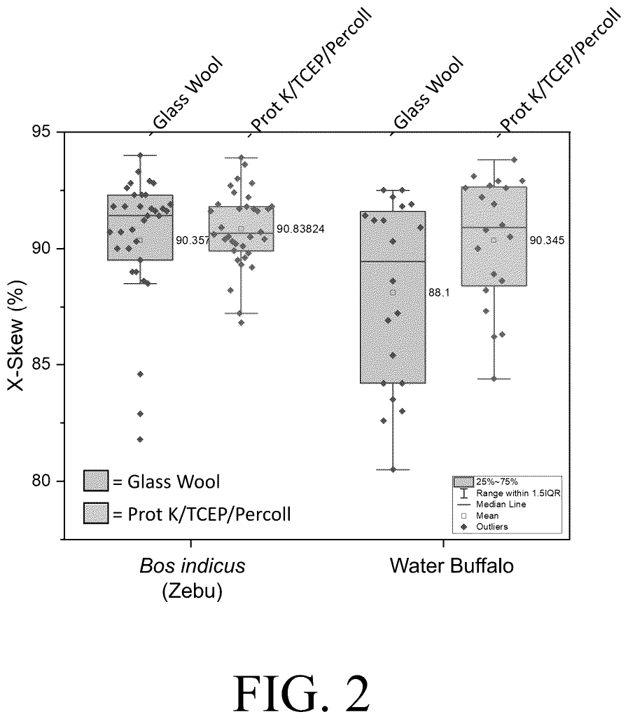

FIG. 2—the present teachings produce better x-skew for the Patan and Mehsana samples and less than a 1% decrease for the Brahma samples. This example illustrates that the present teachings provide purification and x-skew at least as good as the previous gold standard.

### Example 8

This example illustrates reproducibility of results between users.

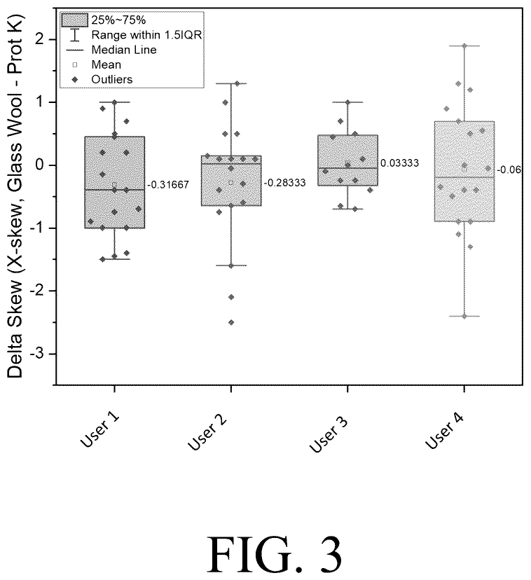

Four users were trained on the techniques described in Example 1-Example 5 and each user processed aliquots of the same samples. The difference between the skew measured and the average historical glass wool skew for ABS were calculated and then plotted for each user as “delta skew;” the plots are shown in FIG. 3. This example illustrates that the methods of the present teachings are reproducible between users.

### Example 9

This example illustrates consistency of skew between glass wool and a method of the present teachings, even when the skew is low.

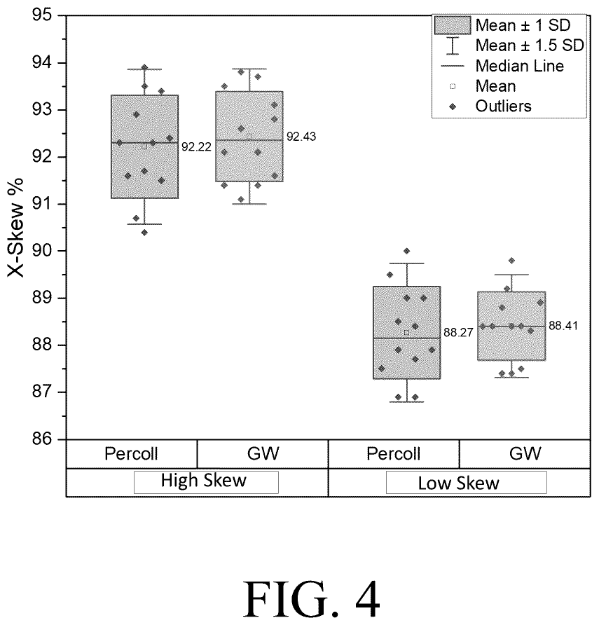

Ejaculates were collected from four bulls. Each ejaculate was split over four processing machines, and three samples were taken from each station. This was done at two different machine settings, with one intentionally producing a lower x-skew. 10 ml of sample was run over glass wool, and 5 ml of sample was run according to Example 3-Example 5. The % of x-skew was plotted and is shown in FIG. 4. This example illustrates consistency between glass wool and a method of the present teachings even when x-skew is low.

### Example 10

This example illustrates treatment of fresh sexed semen with TCEP and proteinase K.

5 ml samples are removed from the output of a sex selection instrument and 1 mL of room temperature 1×Tris buffer is added. The tube is inverted several times to homogenize the 1×Tris and collected throughput. The tubes are then centrifuged for 10 minutes at 300 rcf. The 1×Tris/sex selection buffer mixture is aspirated off of each of the pellets, leaving <5011.1 after aspiration. 300 μl of the treatment buffer (see Example 1) is then added to each of the cell pellets. Each pellet is vortexed to resuspend, and then the tubes are incubated without rotation at 37° C. for 15 minutes.

All publications cited herein are hereby incorporated by reference, each in their entirety.

What is claimed is:

1. A method of removing dead cells from sex selected semen for DNA extraction comprising:
treating non-human mammalian sex selected semen with proteinase K and tris(2-carboxyethyl)phosphine (TCEP) to form treated semen; and
separating dead cells in the treated semen from live cells in the treated semen using a density gradient media.

2. The method of claim 1, wherein the density gradient media is polyvinylpyrrolidone (PVP) coated colloidal silica.

3. The method of claim 1, wherein the density gradient media is silane coated colloidal silica.

4. The method of claim 1, wherein the separating comprises layering the treated semen onto the density gradient media to form a layered sample and then centrifuging the layered sample.

5. The method of claim 1, wherein the non-human mammalian sex selected semen is treated with 0.1-1.5 U/ml proteinase K.

6. The method of claim 1, wherein the non-human mammalian sex selected semen is treated with 0.2-1.2 U/ml proteinase K.

7. The method of claim 1, wherein the non-human mammalian sex selected semen is treated with about 0.75 U/ml proteinase K.

8. The method of claim 1, wherein the non-human mammalian sex selected semen is treated with 1 mM-10 mM TCEP.

9. The method of claim 1, wherein the non-human mammalian sex selected semen is treated with 5-10 mM TCEP.

10. The method of claim 1, wherein the non-human mammalian sex selected semen is treated with about 7.5 mM TCEP.

11. The method of claim 1, wherein the density gradient media is an 85% solution of polyvinylpyrrolidone (PVP) coated colloidal silica.

12. The method of claim 1, wherein the non-human mammalian sex selected semen is a frozen-thawed sample.

13. A method of removing dead cells for assessing skew on sex selected non-human mammalian semen comprising:
treating non-human mammalian sex selected semen with proteinase K and TCEP to form treated semen;
depositing the treated semen onto a density gradient media to form a layered sample;
centrifuging the layered sample to form a supernatant and pellet;
aspirating the supernatant;
washing the density gradient media out of the pellet;
extracting DNA from the pellet; and
performing digital droplet PCR to assess sex skew.

14. The method of claim 13, wherein the density gradient media is an 85% solution of polyvinylpyrrolidone (PVP) coated colloidal silica.

15. The method of claim 13, wherein the non-human mammalian sex selected semen is treated with 0.1-1 U/ml proteinase K.

16. The method of claim 13, wherein the non-human mammalian sex selected semen is treated with 0.3-0.9 U/ml proteinase K.

17. The method of claim 13, wherein the non-human mammalian sex-selected semen is treated with about 0.75 U/ml of proteinase K.

18. The method of claim 13, wherein the non-human mammalian sex selected semen is treated with 1 mM-10 mM TCEP.

19. The method of claim 13, wherein the non-human mammalian sex selected semen is treated with 4-9 mM TCEP.

20. The method of claim 13, wherein the non-human mammalian sex selected semen is treated with about 7.5 mM TCEP.

21. The method of claim 13, wherein:
the non-human mammalian sex selected semen is treated with about 0.5 U/ml of proteinase K and about 7.5 mM TCEP;
the density gradient media is an 85% solution of polyvinylpyrrolidone (PVP) coated colloidal silica; and
the centrifuging is at 1000 RCF for 30 minutes.

22. The method of claim 13, wherein the non-human mammalian sex selected semen is a frozen-thawed sample.

23. A method of assessing a sex skew of sex selected non-human mammalian semen comprising:
treating non-human mammalian sex selected semen with about 0.5 U/ml of proteinase K and 7.5 mM TCEP to form treated semen;
depositing the treated semen onto an 85% solution of polyvinylpyrrolidone (PVP) coated colloidal silica to form a layered sample;
centrifuging the layered sample at 1000 RCF for 30 minutes to form a supernatant and pellet;
aspirating the supernatant;
washing the colloidal silica coated with PVP out of the pellet;
extracting DNA from the pellet; and
performing digital droplet PCR to assess sex skew.
